(19) United States
(12) Patent Application Publication
(10) Pub. No.: US 2016/0302858 A1
(43) Pub. Date: Oct. 20, 2016

(54) TISSUE DIAGNOSIS AND TREATMENT USING ELECTRODES AND MINI-ELECTRODES

(71) Applicant: Boston Scientific Scimed Inc., Maple Grove, MN (US)

(72) Inventor: Robert F. Bencini, Sunnyvale, CA (US)

(21) Appl. No.: 15/130,975

(22) Filed: Apr. 16, 2016

Related U.S. Application Data
(60) Provisional application No. 62/148,857, filed on Apr. 17, 2015.

(51) Int. Cl. A61B 18/14

(52) U.S. Cl. CPC A61B 18/1492; A61B 2018/1467; A61B 2018/00267

(57) ABSTRACT

This disclosure provides design, material, manufacturing method, and use alternatives for medical devices. An example medical device includes an elongate shaft having a proximal portion and a distal portion and an expandable member disposed along the distal portion of the elongate shaft. The expandable member includes one or more electrodes disposed thereon. The medical device further includes an ablation electrode disposed adjacent the expandable member and one or more mini-electrodes adjacent the ablation electrode. Additionally, the ablation electrode, the one or more electrodes and the one or more mini-electrodes are electrically isolated from each other.

### CROSS-REFERENCE TO RELATED APPLICATION

This application claims priority to Provisional Application No. 62/148,857, filed Apr. 17, 2015, which is herein incorporated by reference in its entirety.

### TECHNICAL FIELD

The present disclosure pertains to medical devices, and methods for manufacturing medical devices. More particularly, the present disclosure pertains to elongated intracorporeal medical devices including a tubular member connected with other structures, and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example medical device comprises:
an elongate shaft having a proximal portion and a distal portion; an expandable member disposed along the distal portion of the elongate shaft, wherein the expandable member includes one or more electrodes disposed thereon;
an ablation electrode disposed adjacent the expandable member; and one or more mini-electrodes adjacent the ablation electrode;
wherein the ablation electrode, the one or more electrodes and the one or more mini-electrodes are electrically isolated from each other.

Alternatively or additionally to any of the embodiments above, further comprising a transforming member positioned adjacent the expandable member, wherein the transforming member is designed to shift the expandable member between a first non-expanded configuration, a second expanded configuration, and a third prolapsed configuration.

Alternatively or additionally to any of the embodiments above, wherein the transforming member is coupled to a proximal portion of the expandable member, and wherein the expandable member has a basket shape when in the second configuration.

Alternatively or additionally to any of the embodiments above, wherein the expandable member includes a plurality of splines, wherein the one or more splines have an outwardly facing surface, and wherein one or more electrodes are disposed along the outwardly facing surface.

Alternatively or additionally to any of the embodiments above, wherein the one or more electrodes are positioned on a distal half of the plurality of splines.

Alternatively or additionally to any of the embodiments above, wherein the expandable member includes one or more blade members, wherein the one or more blade members are designed to shift between a first position, a second position and a third position, wherein the blade members are positioned along the elongate shaft when in the first position and third position, and wherein the blade members extend radially outward from the elongate shaft when in the second position.

Alternatively or additionally to any of the embodiments above, the blade members include an outwardly facing surface when in the first position, and wherein the one or more electrodes are disposed along the outwardly facing surface.

Alternatively or additionally to any of the embodiments above, further comprising one or more pairs of electrodes disposed along the distal portion of the elongate shaft, wherein the elongate pairs are designed to operate in a bipolar sensing configuration.

Alternatively or additionally to any of the embodiments above, wherein the mini-electrodes are capable of collecting one or more signals corresponding to tissue viability; wherein the electrodes are capable of collecting one or more signals corresponding to the electrical pathways of the heart adjacent the medical device; and wherein the ablation electrode is capable of applying ablation energy in response to the signals collected by the electrodes and/or mini-electrodes.

Alternatively or additionally to any of the embodiments above, wherein the signals collected by the electrodes and mini-electrodes are collected simultaneously, and wherein the magnitude and duration of ablation energy applied corresponds to the signals collected by the electrodes and mini-electrodes.

Alternatively or additionally to any of the embodiments above, further comprising a processor, wherein the processor is electrically coupled to the electrodes and mini-electrodes, and wherein the processor is designed to simultaneously sense the signals collected by the electrodes and mini-electrodes, and wherein the processor determines the amount of ablation energy applied by the ablation electrode.

Alternatively or additionally to any of the embodiments above, further comprising a display, wherein the display displays diagnostic information corresponding to the signals collected by the mini-electrodes and the electrodes, and wherein the display displays diagnostic information corresponding to the applied ablation energy.

A system for diagnosing and/or treating the heart comprises:
a catheter having a proximal portion and a distal portion;
an ablation electrode disposed along the distal portion of the catheter;
a mini-electrode disposed on the ablation electrode;
a mapping electrode disposed on an expandable member adjacent the ablation electrode;
wherein the mini-electrode is designed to sense a first signal;
wherein the mapping electrode is designed to sense a second signal; and
a processor, wherein the processor is configured to:
   1) process the first signal sensed by the mini-electrode; and
   2) process the second signal sensed by the mapping electrode.

Alternatively or additionally to any of the embodiments above, the expandable member, the expandable member having a plurality of splines, wherein the one or more splines have an outwardly facing surface, and wherein the mapping electrode is disposed along the outwardly facing surface.

Alternatively or additionally to any of the embodiments above, wherein the mapping electrode is positioned on a distal half of the plurality of splines.

Alternatively or additionally to any of the embodiments above, wherein the first signal is electrically isolated from the second signal.

Alternatively or additionally to any of the embodiments above, wherein the first signal sensed by the mini-electrode corresponds to the proximity of the mini-electrode to tissue.

Alternatively or additionally to any of the embodiments above, wherein the catheter further comprises one or more bipolar electrode pairs, and wherein the bipolar electrode pairs are designed to operate in a bipolar sensing configuration.

Alternatively or additionally to any of the embodiments above, further comprising a display, wherein the display displays information corresponding to the signals collected by the mini-electrode and the mapping electrode.

A method for treating the heart comprises:
advancing a catheter to a chamber of the heart, the catheter comprising:
   an ablation electrode;
   an expandable member positioned adjacent the ablation electrode;
   one or more mini-electrodes positioned adjacent the ablation electrode; and
   one or more electrodes positioned adjacent the expandable member;
   expanding the expandable member within the chamber; sensing one or more electrical signals of the heart using the one or more electrodes;
   verifying tissue contact of the ablation electrode using the one or more mini-electrodes; and
   delivering ablation energy through the ablation electrode.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is an example catheter system deployed in the heart;

FIG. 2 illustrates an example medical device in a first configuration;

FIG. 3 illustrates the example medical device of FIG. 2 in a second configuration;

FIG. 4 illustrates the example medical device of FIG. 2 in a third configuration;

FIG. 5 illustrates an alternative medical device in a first configuration.

FIG. 6 illustrates the example medical device of FIG. 5 in a second configuration; and

FIG. 7 illustrates the example medical device of FIG. 5 in a third configuration.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to an embodiment“, some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

The following detailed description should be read with reference to the drawings in which similar elements in different drawings are numbered the same. The drawings, which are not necessarily to scale, depict illustrative embodiments and are not intended to limit the scope of the invention.

Cardiac arrhythmia and/or other cardiac pathology contributing to abnormal heart function may originate in cardiac cellular tissue. One technique that may be utilized to treat the arrhythmia and/or cardiac pathology may include ablation of tissue substrates contributing to the arrhythmia and/or cardiac pathology. The tissue in the substrate may be electrically disrupted, or ablated, by heat, chemicals or other means of creating a lesion in the tissue, or otherwise can be electrically isolated from the normal heart circuit. Electrophysiology therapy involves locating the tissue contributing to the arrhythmia and/or cardiac pathology using an ablation, mapping and/or diagnosing catheter and then using the ablation catheter (or another device) to destroy and/or isolate the tissue.

Prior to performing an ablation procedure, a physician or clinician may utilize specialized mapping and/or diagnostic catheters to precisely locate tissue contributing and/or causing an arrhythmia or other cardiac pathology. Therefore, it may be desirable to precisely locate the targeted tissue prior to performing the ablation procedure in order to effectively alleviate and/or eliminate the arrhythmia and/or cardiac pathology. Further, precise targeting of the tissue may prevent or reduce the likelihood that healthy tissue (located proximate the targeted tissue) is damaged.

Several methods and/or techniques may be employed to precisely locate targeted tissue where an ablation or other therapeutic procedure may be performed. An example method may include advancing a mapping catheter to a cardiac location to map and/or determine adverse electrical pathways of the heart. In some instances, the mapping electrodes may be coupled to a processor. Further, the processor may receive sensed electrical signals from the mapping electrodes and determine a “global” and/or “birds-eye” view of the cardiac electrical activity.

The method may further include removing the mapping catheter and advancing an ablation catheter to a generalized target cardiac location (e.g. a generalized target location determined via the mapping catheter). The ablation catheter may include one or more mini-electrodes and/or ablation electrodes located on a distal portion of the ablation catheter. In some instances, the mini-electrodes may be located on the ablation electrode. The mini-electrodes may sense, measure and/or provide a processor with information relating to electrical activity within the cardiac tissue. Further, the mini-electrodes may be designed to measure and/or sense tissue viability and/or death. For example, a clinician may be able to determine if tissue at or near a treatment site has been ablated (e.g. killed). Additionally, using sensed and/or measured electrical information from cardiac tissue, the processor may be designed to correlate the spatial location of the distal portion of the catheter in relation to the cardiac tissue. For example, the mini-electrodes may measure the impedance, resistance, voltage potential, etc. and determine how far the distal portion of the ablation catheter is to cardiac tissue and/or determine tissue viability.

It may be desirable to ablate the targeted cardiac region based on the electrical information collected by the mapping and/or mini-electrodes. Ablative therapy may be provided by the ablation electrode.

In some examples, application of ablation therapy may create scar tissue and/or an electrical block in targeted cardiac tissue. Furthermore, a physician may be able to monitor the measurement, diagnosis and application of ablative energy in real-time (e.g. on a 3D display showing real-time treatment of targeted cardiac tissue). In some instances, the physician may be able to monitor electrodes (e.g. mini-electrodes) in direct contact with tissue. Additionally, the physician may be able to determine whether to stop and/or alter the applied therapy (e.g. therapeutic procedure) based on the real-time feedback provided from the display. For example, in some examples a physician may apply ablative energy to a target region based on global mapping information. During the application of ablative energy, the physician may observe the necrosis of cellular tissue (e.g. by observing tissue viability information collected from the mini-electrodes), and may alter and/or cease the application of ablative energy based on this information. In some instances, the physician may opt to move the catheter to a location more appropriate for therapy.

Some of the methods and systems disclosed herein may utilize a single therapeutic device capable of performing a range of therapeutic functions. For example, the methods and systems may include utilizing a single catheter including mapping electrodes, mini-electrodes and an ablation electrode to perform a diagnostic and/or therapeutic procedure. Furthermore, the single, therapeutic device may be utilized in combination with a display to treat cardiac tissue in real-time.

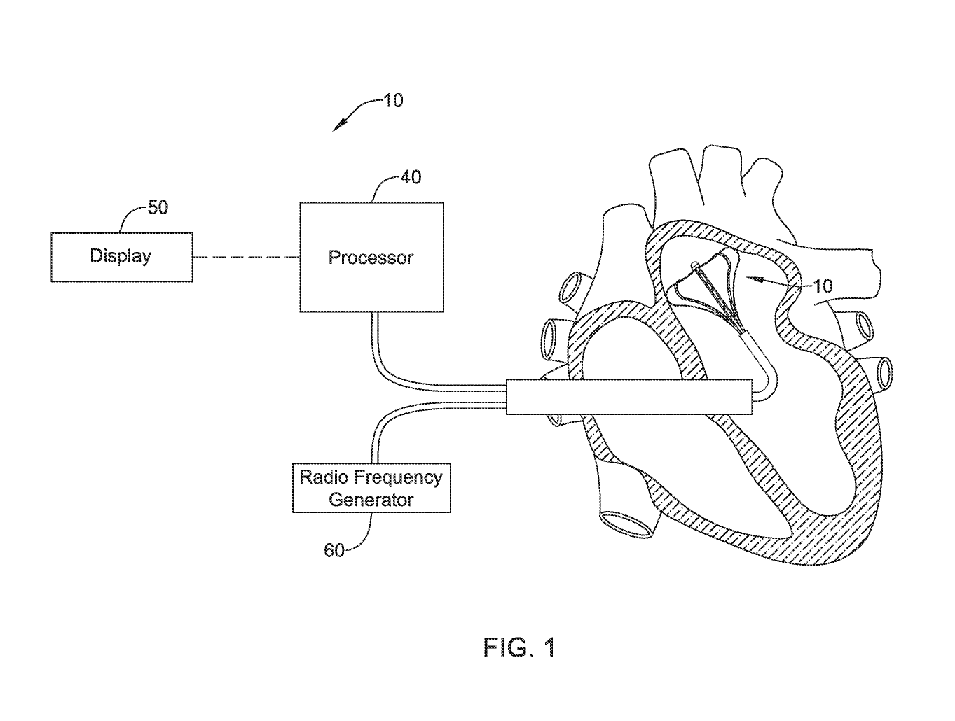

FIG. 1 is a schematic view of a system 10 for accessing a targeted tissue region in the body for diagnostic and/or therapeutic purposes. System 10 may deployed in the left atrium of the heart. Alternatively, system 10 can be deployed in other regions of the heart, such as the left ventricle, right atrium, or right ventricle. While system 10 may be described herein as being used for ablating myocardial tissue, system 10 (and the methods described herein) may alternatively be configured for use in other tissue ablation applications, such as procedures for ablating tissue in the prostate, brain, gall bladder, uterus, nerves, blood vessels and other regions of the body, including in systems that are not necessarily catheter-based.

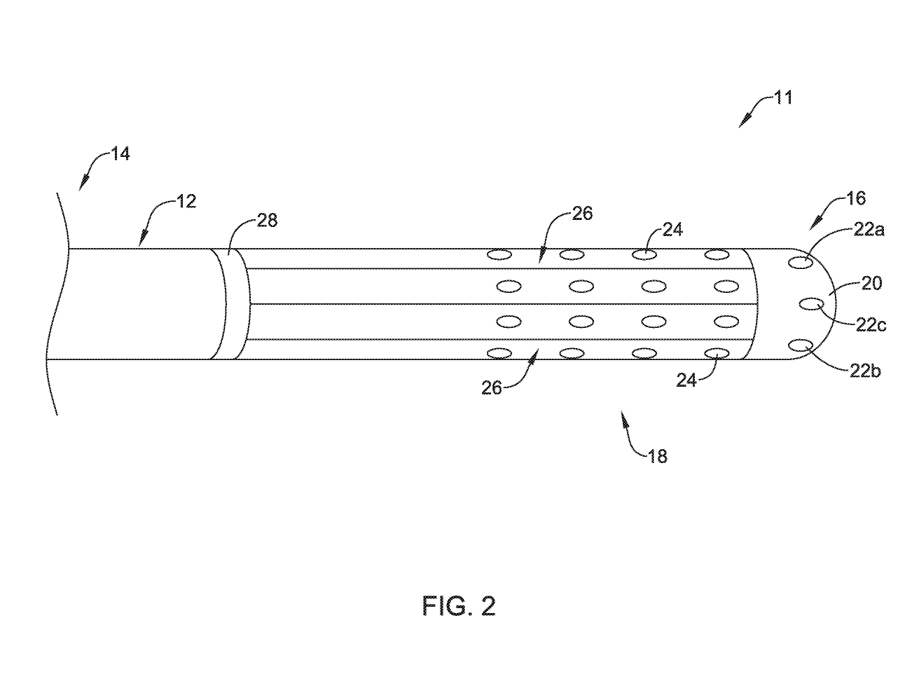

FIG. 2 illustrates an example cardiac mapping and/or ablation system 10 in a non-expanded configuration. As shown in FIG. 2, system 10 may include catheter 11, a processor 40 (e.g., a mapping processor, ablation processor, and/or other processor), a display 50 and a generator 60. Illustratively, catheter 11 may be operatively coupled to at least one or more (e.g., one or both) of processor 40, display 50 and generator 60.

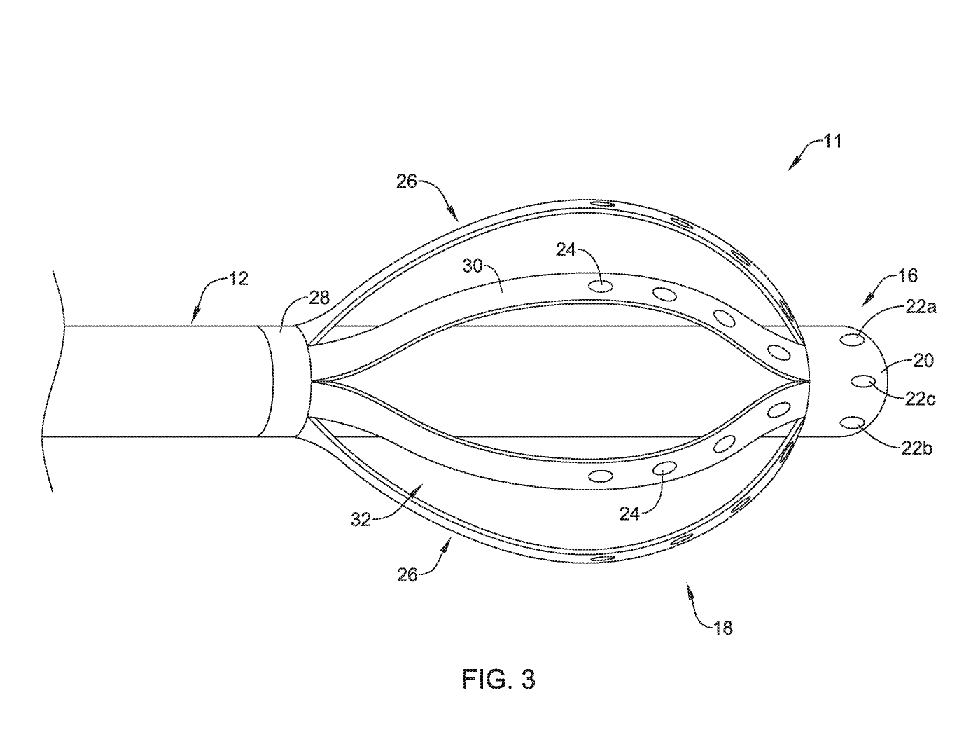

Catheter 11 may include a catheter shaft 12. Shaft 12 may have a proximal end region 14 and a distal end region 16. Distal region 16 may include a three-dimensional expandable member or structure 18 and ablation electrode 20. In the illustrated embodiment, structure 18 may take the form of a basket defining an open interior space 32 (see FIG. 3), although other structures could be used. Structure 18 may include a plurality of mapping electrodes 24 each having an electrode location on structure 18 and a conductive member extending along catheter shaft 12. Electrodes 24 may be configured to sense intrinsic physiological activity in the anatomical region. In some embodiments, the electrodes 24 may be configured to detect global electrical signals of the intrinsic physiological activity within the anatomical structure (e.g., the electrical pathways of cardiac activity).

Electrodes 24 may be electrically coupled to a processing system 40. A signal wire (not shown) may be electrically coupled to each electrode 24 on the basket structure 20. The wires may extend along catheter shaft 12 and electrically couple each electrode 24 to an input of the processing system 40. Electrodes 24 may sense electrical activity in an anatomical region, e.g., myocardial tissue. The sensed activity (e.g., global electrical activity) may be processed by the processing system 40 to assist a physician by generating a map that identifies a site or sites (e.g. diseased tissue) within the heart appropriate for a diagnostic and/or treatment procedure (e.g. an ablation procedure).

The processing system 40 may include dedicated circuitry (e.g., discrete logic elements and one or more microcontrollers; application-specific integrated circuits (ASICs); or specially configured programmable devices, such as, for example, programmable logic devices (PLDs) or field programmable gate arrays (FPGAs)) for receiving and/or processing the acquired activation signals. In some embodiments, the processing system 40 includes a general purpose microprocessor and/or a specialized microprocessor (e.g., a digital signal processor, or DSP, which may be optimized for processing activation signals) that executes instructions to receive, analyze and display information associated with the received activation signals. In such implementations, the processing system 40 can include program instructions, which when executed, perform part of the signal processing. Program instructions can include, for example, firmware, microcode or application code that is executed by microprocessors or microcontrollers. The above-mentioned implementations are merely exemplary, and the reader will appreciate that the processing system 40 can take any suitable form.

The processing system 40 may output to device 50 the display of relevant parameters (e.g. adverse cardiac electrical patterns, tissue viability, tissue pulses, tissue proximity, etc.) for viewing by a physician. In the illustrated embodiment, device 50 is a display, such as a CRT, LED, 3D display, other type of display, or a printer, for example. Device 50 may present the relevant parameters in a format useful to the physician. In addition, the processing system 40 may generate position-identifying output for display on the device 50 that aids the physician in guiding an ablation electrode into contact with tissue at the site identified for ablation. For example, the display 50 may show the electrical pathways of cardiac activity corresponding to one or more electrodes 24 and/or a degree of contact of the distal end region 16 of catheter 11 with tissue. Additionally, the display 50 may show tissue “pulses” (e.g. information corresponding to the viability of the tissue) in response to applied ablative energy, for example. It is contemplated that the elements may be displayed alone or in combination with one another.

The illustrated three-dimensional structure 18 may include flexible splines 26 generally extending in a circumferentially spaced relationship. As discussed herein, the three-dimensional structure 18 may take the form of a basket defining an open interior space 32. In some embodiments the splines 26 are made of a resilient inert material, such as Nitinol, other metals, silicone rubber, suitable polymers, or the like and are connected between a transition member 28 and ablation electrode 20 in a resilient, pretensioned condition, to bend and conform to the tissue surface they contact. In the illustrated embodiment, four splines 26 form the three dimensional structure 18. Additional or fewer splines 26 could be used in other embodiments. As illustrated, each spline 26 carries four mapping electrodes 24. Additional or fewer mapping electrodes 24 could be disposed on each spline 26 in other embodiments of the three dimensional structure 18.

Transition member 28 may be movable along the major axis of catheter 12. For example, moving transition member 28 in a distal direction may cause structure 18 to expand and assume the structure shown in FIG. 2. When in an expanded configuration, moving transition member in a proximal direction may collapse the structure 18 into a compact, low profile condition suitable for introduction into and/or removal from an interior space of an anatomical structure, such as, for example, the heart. While not shown in the figures, it is contemplated that transition member 28 may be coupled to an actuation member (e.g. pull wire, outer shaft). Manipulation of the actuation member may move transition member 28 in a corresponding manner.

FIGS. 2-4 show example basket structure 18 including a plurality of mapping electrodes 24. Basket structure 18 may be a full or partial basket. In the illustrated examples, the basket structure includes sixteen mapping electrodes 24. Mapping electrodes 24 are disposed in groups of four electrodes on each of four splines. While an arrangement of sixteen mapping electrodes 24 is shown disposed on basket structure 20, mapping electrodes 24 may alternatively be arranged in different numbers (more or fewer splines and/or electrodes), on different structures, and/or in different positions. Further, FIGS. 2-4 show electrodes 24 located on the distal half of expandable structure 18. In addition, multiple basket structures can be deployed in the same or different anatomical structures to simultaneously obtain signals from different anatomical structures.

FIG. 3 shows example electrodes 24 disposed along spline 26. In some instances, such as that shown in FIG. 3, one or more of electrodes 24 may be a generally flat shaped, affixed along an outer surface 30 of spline 26. However, it is contemplated that electrodes 24 may be coupled to spline 26 using a variety of methodologies. As discussed herein, electrodes 24 may be described as being “affixed,” “on” and/or otherwise embedded and/or encased on any structure contemplated herein. This is not intended to be limiting. Positioning/locating electrodes 24 along spline 26 may include embedding, partially embedding, encasing, partially encasing, isolating, attaching, affixing, fastening, bonding to the outer surface, embedding within the wall, or the like. Additionally, as shown and described with respect to FIGS. 2-4, it is contemplated that one or more of electrodes 24 may be affixed to splines 26.

While the above examples (and figures) disclose electrodes 24 being positioned on outer surface 30 of spline 26, it is contemplated that electrodes 24 may include different shapes and attachment configurations. For example, electrodes 24 may be ring electrodes (not shown in figures). Ring electrodes may generally wrap around the surface of spline 26. Ring electrodes may be generally cylindrical in shape and substantially surround a given spline 26.

As shown in FIGS. 2-4, ablation electrode 20 may be positioned adjacent expandable member 18. In other examples, ablation electrode 20 may be positioned distal to expandable member 18. Further, FIGS. 2-4 show mini-electrodes 22a-22c disposed on ablation electrode 20. While FIGS. 2-4 show three mini-electrodes 22a-22c, it is contemplated that more or fewer mini-electrodes may be disposed on ablation electrode 20. For example, there may be 1, 2, 3, 4, 5, 6, 7, 8, 9, 10, 15, 20, 50, 75 or 100 mini-electrodes 22a-22c disposed on ablation electrode 20. As discussed above, mini-electrodes 22a-22c may sense and collect localized electrical activity, tissue proximity information and/or tissue viability (e.g. diseased state). In some instances, mini-electrodes 22a-22c may map electrical activity and/or tissue proximity as electrode pairs. For example, electrodes 22a-22c may map as bi-polar electrode pairs 22a-22b, 22b-22c and/or 22a-22c. Additionally, each individual electrode 22a-22c may operate in a unipolar configuration.

The mini-electrodes 22a-22c may sense other activity. For example, it is contemplated that mini-electrodes 22a-22c may monitor tissue pulses corresponding to the viability of the targeted cardiac region. In some instances, the mini-electrodes 22a-22c may operate in a bi-polar configuration to sense and/or monitor tissue pulses (e.g. cellular viability, cellular atrophy and/or cell death).

Mini-electrodes 22a-22c may be positioned such that there is minimal space between adjacent mini-electrodes. The smaller size of mini-electrodes 22a-22c (relative to ablation electrode 20, for example) may allow mini-electrodes 22a-22c to be positioned in multiple configurations. For example, mini-electrodes 22a-22c may circumferentially aligned as shown in FIGS. 2-4. However, it is contemplated that mini-electrodes 22a-22c may be offset in a variety of different configurations. For example, mini-electrodes 22a-22c may be positioned longitudinally along the longitudinal axis of shaft 12 or may be positioned on the apex of ablation electrode 20.

As discussed herein, mini-electrodes 22a-22c may be described as being “on,” “along,” and/or otherwise embedded and/or encased on a given structure. This is not intended to be limiting. Rather, mini-electrodes 22a-22c can be positioned and/or otherwise located at any suitable position/location along the distal end region 16, ablation electrode 20 and/or at other locations along catheter shaft 12. Positioning/locating mini-electrodes 22a-22c may include embedding, partially embedding, encasing, partially encasing, isolating, attaching, affixing, fastening, bonding to the outer surface, embedding within the wall, or the like.

Even though much of the discussion herein has been directed to embodiments in which mini-electrodes 22a-22c have been positioned “on” distal ablation tip electrode 20, it is further contemplated that one or more mini-electrodes 22a-22c may be positioned along the catheter shaft 12 at a position that is away from distal ablation tip electrode 20 and continue to function substantially equivalent to those embodiments in which mini-electrodes are positioned “on” ablation electrode 20.

Additionally, and in some embodiments, mini-electrodes 22a-22c may be electrically isolated from ablation electrode 20. Further, in all embodiments contemplated herein, any given electrode may be electrically isolated from any and/or all other electrodes present in a given catheter system. For example, a layer of insulation (not shown) may be disposed around mini-electrodes 22a-22c. In embodiments where mini-electrodes 22a-22c are disposed along ablation electrode 20, the insulation may insulate mini-electrode 22a-22c from ablation electrode 20. In some embodiments, the insulation may surround and isolate mini-electrode 22a-22c from ablation electrode 20.

Similar to that described above with respect to mapping electrodes 24, mini-electrodes 22a-22c and/or ablation electrode 20 may be electrically coupled to a processing system 40. A signal wire (not shown) may be electrically coupled to each mini-electrode 22a-22c. A separate signal wire (not shown) may be electrically coupled to ablation electrode 20. The wires may extend along catheter shaft 12 and electrically couple each mini-electrode 22a-22c and/or ablation electrode 20 to an input of the processing system 40.

In some embodiments, ablation electrode 20 may be connected to energy generator 60 capable of supplying and/or delivering ablative energy to ablation electrode 20. For example, energy generator 60 may include an RF generator. Additionally, generator 60 may be connected to processor 40. Processor 40 may include processing feedback to generator 60. The processing feedback may include processing instructions which determine the amount of energy delivered by generator 60 to ablation electrode 20.

In some instances, distal end region 16 of catheter 11 may be deflected to position ablation electrode 20 and/or mini-electrodes 22a-22c adjacent target tissue or to position the distal end region 16 of catheter 11 for another suitable purpose. Additionally, or alternatively, distal end region 16 of catheter 11 may have a pre-formed shape adapted to facilitate positioning ablation electrode 20, mini-electrodes 22a-22c and/or electrodes 24 adjacent a target tissue. Illustratively, the preformed shape of distal end region 16 of catheter 11 may be a radiused shape (e.g., a generally circular shape or a generally semi-circular shape) and/or may be oriented in a plane transverse to a general longitudinal direction of shaft 12. These are just examples. Other configurations are contemplated.

As discussed above, electrodes 24 and mini-electrodes 22a-22c may sense electrical activity and/or tissue proximity in an anatomical region, e.g., myocardial tissue. The sensed activity (e.g., global electrical activity, localized electrical activity, tissue proximity) may be processed by the processing system 40 to assist a physician by generating a map that identifies a site or sites (e.g. diseased tissue) within the heart appropriate for a diagnostic and/or treatment procedure (e.g. an ablation procedure).

In some embodiments, it may be desirable to simultaneously collect and process one or more electrical signals from electrodes 24 and/or mini-electrodes 22a-22c. Additionally, some embodiments contemplate utilizing simultaneously collected electrical activity signals and/or tissue information from electrodes 24 and/or mini-electrodes 22a-22c and ablate cardiac tissue based on the collected information.

For example, electrodes 24 may collect and or map the “global” or “birds-eye” electrical activity occurring in a particular cardiac region. Global electrical activity may include adverse electrical patterns. For example, mapping electrodes 24 may sense rotor and/or reentrant electrical pathways. A rotor and/or reentrant electrical pathway may be indicative of diseased tissue (e.g. tissue contributing to adverse cardiac function).

Further, in some examples, once electrodes 24 determine generalized area of diseased tissue and/or adverse cardiac electrical patterns, mini-electrodes 22a-22c may determine localized cardiac electrical activity corresponding to a particular target region. For example, mini-electrodes 22a-22c may sense and/or collect electrical information from cardiac tissue contributing (or causing) adverse cardiac function (e.g. arrhythmia). For example, mini-electrodes 22a-22c may collect information relating the viability of tissue function and/or the monitoring of cellular death as ablation (e.g. RF) energy is applied. Further, mini-electrodes 22a-22c may determine the proximity of the ablation electrode and/or the distal end region 16 to a target site.

After determining a target site, ablation electrode 20 may deliver ablative energy to the target tissue. The amount of ablative energy delivered may correspond to the sensed, monitored and/or collected information from electrodes 24 and mini-electrodes 22a-22c. For example, a physician may monitor (on a 3D display) the real-time effect that the application of ablation energy has on the targeted cellular tissue. Further, the physician may alter and/or cease the application of ablation energy based on the feedback information. For example, a physician may apply RF energy while simultaneously observing the change or response of the target tissue via global electrical information (e.g. electrical pathway information), localized tissue information (e.g. tissue viability, death) or a combination of local and global electrical information. The physician may iteratively and/or repeatedly apply energy and monitor the tissue response over a period of time until the desired tissue response and/or electrical pathways/patterns are achieved.

Additionally, it can be appreciated that a physician and/or clinician may not need to exchange and/or insert multiple catheters while performing the diagnostic and/or therapeutic procedure. Rather, a single catheter (e.g. expandable member 18) may be inserted and utilized to perform global mapping, localized mapping/sensing and ablation procedures.

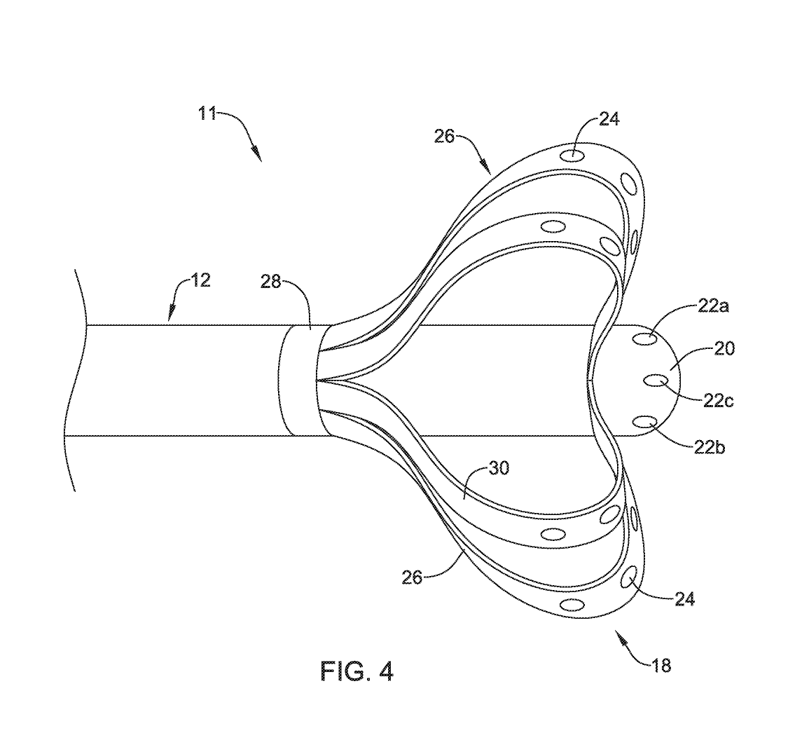

FIG. 4 shows expandable structure 18 in a prolapsed configuration. Actuation of transition member 28 in a distal direction may move expandable structure 18 from the basket-like configuration (shown in FIG. 3) to the prolapsed configuration shown in FIG. 4. The prolapsed configuration depicted in FIG. 4 allows electrodes 24 to move to a more “forward-facing” configuration as compared to their positioned shown in FIG. 3. The forward-facing configuration of FIG. 4 may permit a greater number of electrodes 24 to be placed against cardiac tissue as the expandable structure 18 (including electrodes 24), mini-electrodes 22a-22c and ablation electrode 20 is advanced up against targeted cardiac tissue. Further, as show in FIG. 4, the prolapsed configuration may position ablation electrode 20 (including mini-electrodes 22a-22c) at or near the center of a group of electrodes 24 (e.g. electrodes 24 may be substantially uniformly distributed around ablation electrode 20). It can be appreciated that this configuration allows electrodes 24 to sense the global electrical signals of a cardiac tissue region surrounding mini-electrodes 22a-22c and ablation electrode 20.

It is contemplated that a clinician may move structure 18 between the basket-like configuration and the prolapsed configuration as needed. For example, while probing/mapping the heart chamber, a clinician may utilize structure 18 in the basket-like configuration of FIG. 3. Further, elongate structure 18 may be moved to the configuration shown in FIG. 4 once a particular region of the cardiac chamber is identified for further inspection.

Based, at least in part, on the processed output from electrodes 24, mini-electrodes 22a-22c and/or ablation electrode 20, processor 40 may generate an output to a display 50 for use by a physician or other user. In instances where an output is generated to display 50 and/or other instances, processor 40 may be operatively coupled to or otherwise in communication with display 50. Illustratively, display 50 may include various static and/or dynamic information related to the use of system 10. In one example, the display 50 may include one or more of an image of the target area, an image of shaft 12, an image of expandable structure 18 and/or indicators conveying information corresponding to tissue proximity, which may be analyzed by the user and/or by processor 40 of system 10 to determine the existence and/or location of arrhythmia substrates within the heart, to determine the location of shaft 12 within the heart, an image of expandable structure 18 and/or to make other determinations relating to use of shaft 12 and/or other elongated members.

Further, as discussed above, display 50 may provide “real-time” feedback of the effect that ablating a particular cardiac tissue region has on the overall cardiac function. For example, display 50 may show a cardiac region prior to applying ablative energy and the same region after applying ablative energy. The comparison may indicate whether continued ablation is necessary, or further, indicate whether the adverse cardiac function (e.g. arrhythmia) has been alternated and/or alleviated.

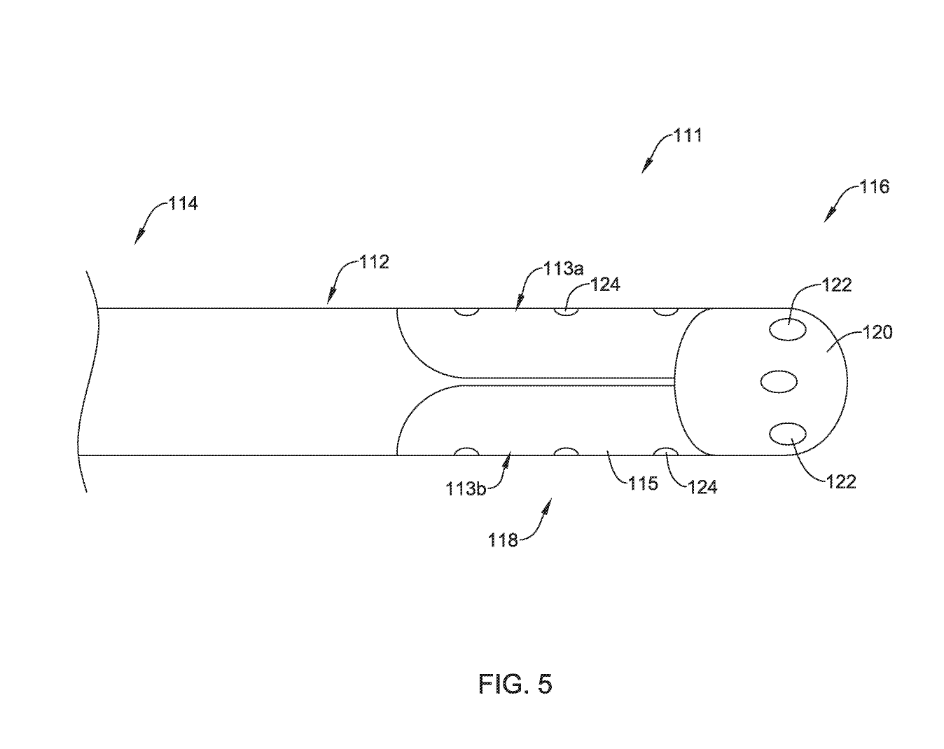

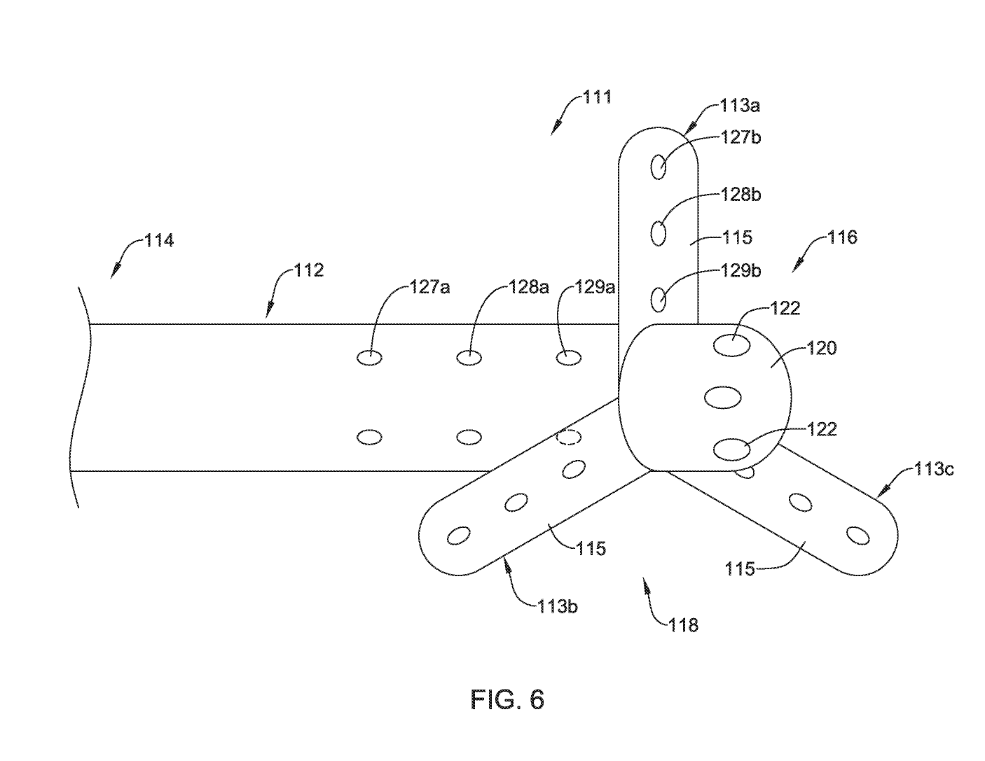

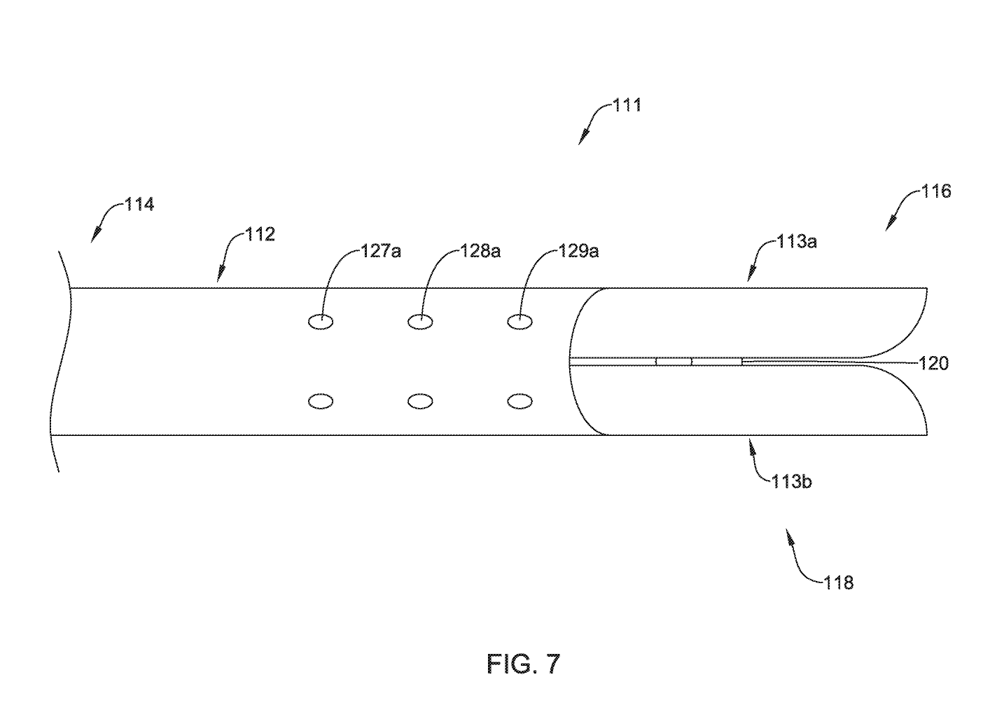

FIG. 5 illustrates an alternative embodiment of catheter system 10 described above. Specifically, FIG. 5 shows catheter system 110 having one or more blade members 113 disposed along catheter shaft 112 in an unexpanded configuration. In some embodiments blade members 113 are made of a resilient inert material, such as Nitinol, other metals, silicone rubber, suitable polymers, or the like and may be coupled to an actuation member (not shown). The actuation member may move blade members 113 between a first or unexpanded configuration (shown in FIG. 5), a second or expanded configuration (shown in FIG. 6) and a third or prolapsed configuration (shown in FIG. 7).

Blade members 113 may include electrodes 124 affixed to outer blade surface 115. As discussed herein, electrodes 124 may be described as being “on,” “along,” and/or otherwise embedded and/or encased on blade members 113. This is not intended to be limiting. Positioning/locating electrodes 124 along blade members 113 may include embedding, partially embedding, encasing, partially encasing, isolating, attaching, affixing, fastening, bonding to the outer surface 115, embedding within the wall, or the like.

FIG. 6 shows catheter 111 in an expanded configuration. As shown, expandable member 118 includes blade members 113a, 113b and 113c. It is contemplated that expandable member 118 may include more or fewer than three blade members 113a-113c. FIG. 6 shows blade members 113a-113c extending outwardly from catheter shaft 112. Further, blade members may have mapping electrodes disposed along outer blade surface 115 of blade members 113a-113c.

Additionally, FIG. 6 shows bipolar electrode pairs 127a/127b, 128a/128b and 129a/129b. In FIG. 6, electrodes 127a, 128a and 129a are shown disposed along catheter shaft 112. Further, electrodes 127b, 128b and 129b are shown affixed on the outer blade surface 115. It is contemplated that more than one pair of bipolar electrodes may be disposed along catheter shaft 112 and blade member 113a-113c. For example, FIG. 6 shows three pairs of bipolar electrode pairs. More or fewer bipolar electrode pairs are contemplated. Furthermore, in some embodiments contemplated herein, the operation of the bipolar electrode pairs shown in FIG. 6 operates substantially similar to the mapping electrodes 24 discussed with respect to FIG. 3.

FIG. 7 shows expandable member 118 in a prolapsed configuration. In the prolapsed configuration, blade members 113 are shown folded distally over ablation electrode 120.

Alternative embodiment 112 may include an ablation electrode 120 and mini-electrodes 122 disposed thereon. The design and operation of ablation electrode 120 and mini-electrodes 122 may be similar to that of ablation electrode 20 and mini-electrodes 22 described above with respect to FIGS. 5-7.

It is contemplated for all embodiments described herein that a given electrode type (e.g. mapping electrode, bipolar electrode, mini-electrode) may be positioned on any catheter structure defined and described herein. For example, while mapping electrodes are described herein as being along an expandable member, it is contemplated that the mapping electrodes, in some alternative embodiments, may be disposed on the catheter shaft and/or an ablation electrode. Further, ablation electrodes, mini-electrodes and/or bipolar electrodes may be positioned on alternative catheter structures and perform as described herein.

Additionally, any of the catheter systems described herein may include additional sensors, ports and/or electrodes. For example, it is contemplated that the embodiments described herein may include one or more fluid ports (e.g. open irrigated), temperature sensors, pressure sensors, array sensors or the like. Further, embodiments are contemplated that use one or combinations of fluid ports, temperature sensors, pressure sensors, array sensors or the like.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

I claim:

1. A medical device, comprising:
an elongate shaft having a proximal portion and a distal portion;
an expandable member disposed along the distal portion of the elongate shaft, wherein the expandable member includes one or more electrodes disposed thereon;
an ablation electrode disposed adjacent the expandable member; and
one or more mini-electrodes adjacent the ablation electrode;
wherein the ablation electrode, the one or more electrodes and the one or more mini-electrodes are electrically isolated from each other.

2. The medical device of claim 1, further comprising a transforming member positioned adjacent the expandable member,
wherein the transforming member is designed to shift the expandable member between a first non-expanded configuration, a second expanded configuration, and a third prolapsed configuration.

3. The medical device of claim 2, wherein the transforming member is coupled to a proximal portion of the expandable member, and
wherein the expandable member has a basket shape when in the second configuration.

4. The medical device of claim 1, wherein the expandable member includes a plurality of splines, wherein the one or more splines have an outwardly facing surface, and wherein one or more electrodes are disposed along the outwardly facing surface.

5. The medical device of claim 4, wherein the one or more electrodes are positioned on a distal half of the plurality of splines.

6. The medical device of claim 1, wherein the expandable member includes one or more blade members, wherein the one or more blade members are designed to shift between a first position, a second position and a third position,
wherein the blade members are positioned along the elongate shaft when in the first position and third position, and
wherein the blade members extend radially outward from the elongate shaft when in the second position.

7. The medical device of claim 6, wherein the blade members include an outwardly facing surface when in the first position, and wherein the one or more electrodes are disposed along the outwardly facing surface.

8. The medical device of claim 6, further comprising one or more pairs of electrodes disposed along the distal portion of the elongate shaft, wherein the elongate pairs are designed to operate in a bipolar sensing configuration.

9. The medical device of claim 1, wherein the mini-electrodes are capable of collecting one or more signals corresponding to tissue viability;
wherein the electrodes are capable of collecting one or more signals corresponding to the electrical pathways of the heart adjacent the medical device; and
wherein the ablation electrode is capable of applying ablation energy in response to the signals collected by the electrodes and/or mini-electrodes.

10. The medical device of claim 9, wherein the signals collected by the electrodes and mini-electrodes are collected simultaneously, and
wherein the magnitude and duration of ablation energy applied corresponds to the signals collected by the electrodes and mini-electrodes.

11. The medical device of claim 10, further comprising a processor, wherein the processor is electrically coupled to the electrodes and mini-electrodes, and
wherein the processor is designed to simultaneously sense the signals collected by the electrodes and mini-electrodes, and
wherein the processor determines the amount of ablation energy applied by the ablation electrode.

12. The medical device of claim 11, further comprising a display, wherein the display displays diagnostic information corresponding to the signals collected by the mini-electrodes and the electrodes, and wherein the display displays diagnostic information corresponding to the applied ablation energy.

13. A system for diagnosing and/or treating the heart, comprising:
a catheter having a proximal portion and a distal portion;
an ablation electrode disposed along the distal portion of the catheter;
a mini-electrode disposed on the ablation electrode;
a mapping electrode disposed on an expandable member adjacent the ablation electrode;
wherein the mini-electrode is designed to sense a first signal;
wherein the mapping electrode is designed to sense a second signal; and
a processor, wherein the processor is configured to:
1) process the first signal sensed by the mini-electrode; and
2) process the second signal sensed by the mapping electrode.

14. The system of claim 13, wherein the expandable member, the expandable member having a plurality of splines, wherein the one or more splines have an outwardly facing surface, and wherein the mapping electrode is disposed along the outwardly facing surface.

15. The system of claim 14, wherein the mapping electrode is positioned on a distal half of the plurality of splines.

16. The system of claim 13, wherein the first signal is electrically isolated from the second signal.

17. The system of claim 13, wherein the first signal sensed by the mini-electrode corresponds to the proximity of the mini-electrode to tissue.

18. The system of claim 13, wherein the catheter further comprises one or more bipolar electrode pairs, and wherein the bipolar electrode pairs are designed to operate in a bipolar sensing configuration.

19. The medical device of claim 13, further comprising a display, wherein the display displays information corresponding to the signals collected by the mini-electrode and the mapping electrode.

20. A method for treating the heart, the method comprising:
advancing a catheter to a chamber of the heart, the catheter comprising:
an ablation electrode;
an expandable member positioned adjacent the ablation electrode; one or more mini-electrodes positioned adjacent the ablation electrode; and
one or more electrodes positioned adjacent the expandable member;
expanding the expandable member within the chamber;
sensing one or more electrical signals of the heart using the one or more electrodes;
verifying tissue contact of the ablation electrode using the one or more mini-electrodes; and
delivering ablation energy through the ablation electrode.
